(19) United States
(12) Patent Application Publication
(10) Pub. No.: US 2024/0197739 A1
(43) Pub. Date: Jun. 20, 2024

(54) Combination Comprising Ribociclib and Amcenestrant

(71) Applicant: Sanofi, Paris (FR)

(72) Inventors: Monsif Bouaboula, Bridgewater, NJ (US); Fangxian Sun, Bridgewater, NJ (US)

(73) Assignee: Sanofi, Paris (FR)

(21) Appl. No.: 18/286,510

(22) PCT Filed: Apr. 12, 2022
(86) PCT No.: PCT/EP2022/059700

§ 371 Date: Oct. 11, 2023

(30) Foreign Application Priority Data
Apr. 12, 2021 (EP) 21315064.2

(51) Int. Cl. A61K 31/519; A61K 31/40; A61P 35/00

(52) U.S. Cl. CPC A61K 31/519; A61K 31/40; A61P 35/00

(57) ABSTRACT

Herein are provided a combination of ribociclib, or a pharmaceutically acceptable salt thereof, such as ribociclib succinate, and amcenestrant, or a pharmaceutically acceptable salt thereof, a pharmaceutical composition containing such a combination, and the therapeutic uses thereof, in particular for the treatment of cancer, including breast cancer.

Herein are provided a combination of ribociclib and amcenestrant, a pharmaceutical composition containing such combination, and the therapeutic uses of such combination and pharmaceutical composition, in particular for the treatment of cancer.

The estrogen receptor α(ESR1) is expressed in the majority of breast tumors, enabling them to respond to the mitogenic actions of estrogens.

Amcenestrant, INN name for the compound 6-(2,4-dichlorophenyl)-5-[4-[(3S)-1-(3-fluoropropyl)pyrrolidin-3-yl]oxyphenyl]-8,9-dihydro-7H-benzo[7]annulene-2-carboxylic acid (also known by its laboratory code SAR439859), is a selective estrogen receptor degrader (SERD) which is an estrogen receptor antagonist and accelerates the proteasomal degradation of the estrogen receptor. This compound is disclosed in the patent application WO 2017/140669:

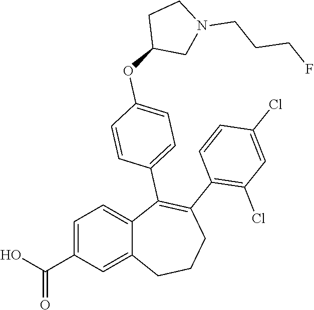 [SMILES: O=C(O)c1ccc2c(c1)CCCC(c1ccc(Cl)cc1Cl)=C2c1ccc(O[C@H]2CCN(CCCF)C2)cc1]

Ribociclib (INN name), or 7-cyclopentyl-N,N-dimethyl-2-[[5-(1-piperazinyl)-2-pyridinyl]amino]-7H-pyrrolo[2,3-d]pyrimidine-6-carboxamide, is a CDK4/6 inhibitor of the following formula:

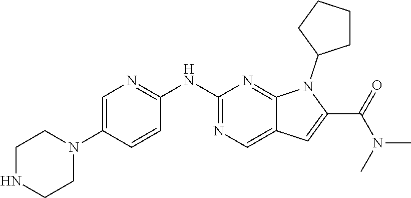 [SMILES: CN(C)C(=O)c1cc2cnc(Nc3ccc(N4CCNCC4)cn3)nc2n1C1CCCC1]

Ribociclib under its succinate form (salt formed with butanedioic acid) is a marketed drug (tradename KISQALI®), see formula below:

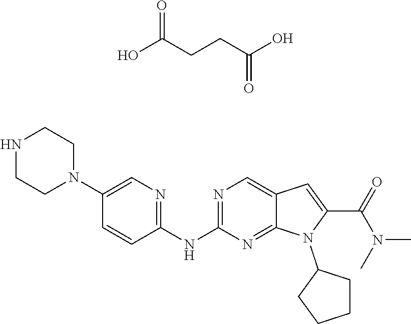 [SMILES: O=C(O)CCC(=O)O ; CN(C)C(=O)c1cc2cnc(Nc3ccc(N4CCNCC4)cn3)nc2n1C1CCCC1]

KISQALI® is indicated in combination with:
an aromatase inhibitor for the treatment of pre/perimenopausal or postmenopausal women with hormone receptor (HR)-positive, human epidermal growth factor receptor 2 (HER2)-negative advanced, or metastatic breast cancer, as initial endocrine-based therapy; or
fulvestrant for the treatment of postmenopausal women with HR-positive, HER2-negative advanced, or metastatic breast cancer, as initial endocrine-based therapy or following disease progression on endocrine therapy.

There is always a need to find new antitumoral treatments. Now, it is shown herein that a combination of amcenestrant with ribociclib is well tolerated, demonstrates significant anti-tumor efficacy, and induces tumor statis, with a synergistic effect compared to each of the active ingredient alone.

Herein is provided a combination comprising amcenestrant and ribociclib.

In the combination provided herein, amcenestrant may exist not only in the form of a zwitterion (i.e., a globally neutral molecule having an acid group and a basic group), but also in the form of addition salts with acids or bases. Such addition salts may be used in the above combination. Also, ribociclib may exist in the form of salts, more particularly of pharmaceutically acceptable salts, in particular the succinate salt. Hence, herein is provided a combination comprising amcenestrant, or a pharmaceutically acceptable salt thereof, and ribociclib, or a pharmaceutically acceptable salt thereof, such as ribociclib succinate.

In an embodiment, the combination of amcenestrant, or a pharmaceutically acceptable salt thereof, with ribociclib, or a pharmaceutically acceptable salt thereof, such as ribociclib succinate, shows therapeutic synergy. A combination demonstrates therapeutic synergy if its therapeutic effect is superior compared to the cumulative effect of either active agent of the combination alone.

In another embodiment, amcenestrant, or a pharmaceutically acceptable salt thereof, and ribociclib, or a pharmaceutically acceptable salt thereof, such as ribociclib succinate, are administered orally.

Provided herein is also a combination of amcenestrant, or a pharmaceutically acceptable salt thereof, and ribociclib, or a pharmaceutically acceptable salt thereof, such as ribociclib succinate, for its use as a medicament.

Provided herein is also a pharmaceutical composition comprising amcenestrant, or a pharmaceutically acceptable salt thereof, and ribociclib, or a pharmaceutically acceptable salt thereof, such as ribociclib succinate, as well as at least one pharmaceutically acceptable excipient.

The excipients are selected from the customary excipients which are known to a person skilled in the art. More particularly, the excipients are selected from those useful for oral administration in whatever form (liquid solution, dispersion or suspension, tablets, capsules, or the like).

In another embodiment, amcenestrant, or a pharmaceutically acceptable salt thereof, and ribociclib, or a pharmaceutically acceptable salt thereof, such as ribociclib succinate, may be administered simultaneously, separately, or spaced out over a period of time (sequential administration). Therefore, the combination and pharmaceutical composition provided herein are not exclusively limited to the ones which are obtained by physical association of the constituents in a single unit dosage, but also to those which allow a separate administration, which can be simultaneous or sequential (also called “spaced out” or “spread out”) over a period of time.

Herein is also provided a pharmaceutical kit which comprises:
(i) a first pharmaceutical composition comprising amcenestrant, or a pharmaceutically acceptable salt thereof, and at least one pharmaceutically acceptable excipient;
(ii) a second pharmaceutical composition comprising ribociclib, or a pharmaceutically acceptable salt thereof, such as ribociclib succinate, and at least one pharmaceutically acceptable excipient;

wherein the first pharmaceutical composition and the second pharmaceutical composition are in separate compartments and are intended to be independently administered, each administration with regards to the other one being simultaneous or spaced out (sequential) over time.

In the combinations, pharmaceutical compositions, and pharmaceutical kit described above, amcenestrant, or a pharmaceutically acceptable salt thereof, and ribociclib, or a pharmaceutically acceptable salt thereof, such as ribociclib succinate, are advantageously present at effective doses, adapted considering the treated pathology and the condition of the patient to which it is administered. In particular, for ribociclib, the recommended dose for adult patients as per KISQALI label is 600 mg (expressed as ribociclib fee base from) once daily, taken orally.

Herein is also provided a combination comprising amcenestrant, or a pharmaceutically acceptable salt thereof, and ribociclib, or a pharmaceutically acceptable salt thereof, such as ribociclib succinate, as well as a pharmaceutical composition and kit as described above, for use in the treatment of cancer.

Herein is also provided amcenestrant, or a pharmaceutically acceptable salt thereof, for use in the treatment of cancer by co-administration with ribociclib, or a pharmaceutically acceptable salt thereof, such as ribociclib succinate.

Herein is also provided ribociclib, or a pharmaceutically acceptable salt thereof, such as ribociclib succinate, for use in the treatment of cancer by co-administration with amcenestrant, or a pharmaceutically acceptable salt thereof.

Co-administration is understood herein as an administration of the active ingredients to a patient in need thereof, which is separated, simultaneous, or spaced out (sequential) over time, with respect to each of the active ingredients.

In some embodiments, amcenestrant, or a pharmaceutically acceptable salt thereof, and ribociclib, or a pharmaceutically acceptable salt thereof, such as ribociclib succinate, are administered in a therapeutically effective amount. A “therapeutically effective amount” means the amount of an active ingredient or combination of active ingredients that, when administered to a patient for treating a disease, is sufficient to affect such treatment for the disease. The “therapeutically effective amount” will vary depending on the disease and its severity and the age, weight, etc., of the subject to be treated.

In some embodiments, amcenestrant, or a pharmaceutically acceptable salt thereof, and ribociclib, or a pharmaceutically acceptable salt thereof, such as ribociclib succinate, are administered in an amount to show therapeutic synergy.

In another embodiment, the cancer is a hormone dependent cancer.

In another embodiment, the cancer is an estrogen receptor dependent cancer, particularly the cancer is an estrogen receptor a dependent cancer.

Herein is also provided a method of treating the pathological conditions indicated above, particularly breast cancer, comprising administering to a subject in need thereof a therapeutically effective amount of amcenestrant, or a pharmaceutically acceptable salt thereof, and a therapeutically effective amount of ribociclib, or a pharmaceutically acceptable salt thereof, such as ribociclib succinate.

Herein is also provided a method of treating the pathological conditions indicated above, particularly breast cancer, comprising administering to a subject in need thereof a pharmaceutical composition or a pharmaceutical kit as described above.

Herein is also provided a method of treating the pathological conditions indicated above, particularly breast cancer, comprising administering to a subject in need thereof a combination as described above.

Herein is also provided a method of treating the pathological conditions indicated above, particularly breast cancer, comprising co-administering to a subject in need thereof amcenestrant, or a pharmaceutically acceptable salt thereof, and ribociclib, or a pharmaceutically acceptable salt thereof, such as ribociclib succinate. In said method, amcenestrant, or a pharmaceutically acceptable salt thereof, is administered with ribociclib, or a pharmaceutically acceptable salt thereof, such as ribociclib succinate, either separately, simultaneously or spaced out over time.

Herein is also provided a method of treating the pathological conditions indicated above, particularly breast cancer, comprising co-administering to a subject in need thereof ribociclib, or a pharmaceutically acceptable salt thereof, such as ribociclib succinate, and amcenestrant, or a pharmaceutically acceptable salt thereof. In said method, ribociclib, or a pharmaceutically acceptable salt thereof, such as ribociclib succinate, is administered with amcenestrant, or a pharmaceutically acceptable salt thereof, either separately, simultaneously or spaced out over time.

Herein is also provided a method of treating cancer comprising administering to a patient in need thereof a therapeutically effective amount of amcenestrant, or a pharmaceutically acceptable salt thereof, in combination with a therapeutically effective amount of ribociclib, or a pharmaceutically acceptable salt thereof, such as ribociclib succinate.

Herein is also provided a method of treating cancer in a patient who is on therapy with compound amcenestrant, or a pharmaceutically acceptable salt thereof, comprising administering to said patient an effective amount of ribociclib, or a pharmaceutically acceptable salt thereof, such as ribociclib succinate.

Herein is also provided a method of treating cancer in a patient on stable treatment with compound amcenestrant, or a pharmaceutically acceptable salt thereof, comprising administering to said patient a therapeutically effective amount of ribociclib, or a pharmaceutically acceptable salt thereof, such as ribociclib succinate.

Herein is also provided a method of treating cancer comprising administering to a patient in need thereof a therapeutically effective amount of compound ribociclib, or a pharmaceutically acceptable salt thereof, such as ribociclib succinate, wherein said patient is also on therapy with amcenestrant or a pharmaceutically acceptable salt thereof.

In an embodiment of the methods described above, the subject is a mammal. In another embodiment, the subject is a human.

Herein is also provided a combination comprising amcenestrant, or a pharmaceutically acceptable salt thereof, and ribociclib, ribociclib, or a pharmaceutically acceptable salt thereof, such as ribociclib succinate, for the manufacture of a medicament useful in treating the pathological conditions indicated above, particularly breast cancer.

Herein is also provided the use of amcenestrant, or a pharmaceutically acceptable salt thereof, in the manufacture of a medicament useful in treating the pathological conditions indicated above, particularly breast cancer, by co-administration with ribociclib or a pharmaceutically acceptable salt thereof, such as ribociclib succinate.

Herein is also provided the use of ribociclib, or a pharmaceutically acceptable salt thereof, such as ribociclib succinate, in the manufacture of a medicament useful in treating the pathological conditions indicated above, particularly breast cancer, by co-administration with amcenestrant or a pharmaceutically acceptable salt thereof.

Herein is also provided an article of manufacture, a packaging, or an administration unit, comprising:
a packaging material;
the above defined combination, pharmaceutical composition, or pharmaceutical kit; and
a label or package insert contained within said packaging material, indicating that said combination, pharmaceutical composition, or pharmaceutical kit is administered to a patient for the treatment of cancer.

The examples below show the pharmacological results obtained with amcenestrant, ribociclib succinate, and their combination against a breast cancer cell line xenograft in mice.

### Evaluation of the Efficacy of Amcenestrant Combined With Ribociclib Against a Subcutaneous Breast Cancer Cell Line Xenograft in Female Nude Mice

In the present study, the anti-tumor efficacy of amcenestrant combined with ribociclib succinate (in the experimental section hereunder, merely referred to as “ribociclib” for conciseness) was investigated after 22 days treatment against a subcutaneous MCF7-Y537S human breast cancer cell line xenograft in female nude mice.

The treated groups included amcenestrant at 20 mg/kg alone, ribociclib at 100 mg/kg alone (weight expressed based on ribociclib free base form), and the combination of amcenestrant and ribociclib at the same dose and regime.

For 22 days, amcenestrant was orally dosed twice a day (BID), and ribociclib was orally dosed once a day (QD). Anti-tumor efficacy was evaluated by tumor volume measurement.

### 1: Experimental Procedure

### 1-1: Animals, Cell Line, Compounds

Female BALB/c nude mice were obtained from Shanghai Sino-British SIPPR/BK Laboratory Animal Co., LTD (Shanghai, CHINA). Animals were allowed to acclimate for at least four days before the study enrollment. Mice were 6 to 8 weeks old and weighed between 18 and 24 grams at the beginning of the treatments. These animals were housed under conditions outlined in the guidelines approved by the Institutional Animal Care and Use Committee (IACUC) of WuXi AppTec following the guidance of the Association for Assessment and Accreditation of Laboratory Animal Care (AAALAC).

Parental MCF7 cells were obtained from the American Type Culture Collection (ATCC® HTB-22™). MCF7-Y537S (ESR1) cell line was MCF7 cells expressing the ER. Y537S variant that was generated by Sanofi Biology Discovery Group. Y537S mutation was introduced in ESR1 construct (GenBank NM_000125.3) by site directed mutagenesis (Toy W. et al., Cancer Discovery, 2017, 7, 277-287). The construct was transfected in MCF7 cells, which were selected for their growth in absence of estradiol. MCF-Y537S is an ESR1 mutation that confers estrogen-independent activity to ERα(Estrogen Receptor alpha) and contributes to endocrine resistant disease (Robinson D.R. et al., Nat Genet., 2013, 45 (12), 1446-1451). The cells were grown in Eagle's Minimum Essential Medium (EMEM) supplemented with 10% fetal bovine serum (FBS), human Insulin, in 5% CO₂ at 37° C. The cells were harvested in 0.25% Trypsin EDTA and washed by Phosphate Buffered Saline (PBS) and re-suspended in PBS with 75% Matrigel. The cells (20×10⁶ cells/per mouse) were subcutaneously (SC) implanted into the right flank of female nude mice.

When the MCF7-Y537S tumors were established, the tumors were reserved as tumor stocks for fragment implantation. The tumors were serially propagated through fragment tissue transplantation subcutaneously. The fragment tumor tissues were subcutaneously implanted into the right flank of female nude mice. Twenty-eight mice were assigned in this experiment.

Ribociclib (Manufacturer: Sanofi; Lot number: VAC.DLE20.41.1; succinate salt) was formulated in 40% SBE-β-CD (sulfobutylether β-cyclodextrin) in water (pH 3).

Dose volume for amcenestrant and ribociclib for oral administration: 10 ml/kg.

Doses: amcenestrant at 20 mg/kg and ribociclib at 100 mg/kg in the above volume.

Concentrations of ribociclib are expressed herein on the basis of its free base form.

### 1-2: Study Design, End Points

The animals required for experiment (plus extra) were pooled and implanted with MCF7-Y537S tumor fragment tissues. On day 0 (20 days post implantation), the mice were pooled and randomly distributed to the treatment and control groups (7 mice per group), where median tumor volumes for each group was 173 mm³. Treatments of amcenestrant and ribociclib were initiated on day 0. For 22 days, amcenestrant was orally administered at 20 mg/kg BID (8 hours apart), and ribociclib was orally administered at 100 mg/kg QD. Animal body weight was assessed daily.

The dosages are expressed in mg/kg and based on daily body weight per animal. Vehicle treated animals were used as controls. Mice were checked daily for adverse clinical reactions. Individual mice were weighed daily until the end of the experiment. Mice would be euthanized when morbid or weight loss ≥20% was observed. Tumors were measured with a caliper twice weekly until final sacrifice. When a tumor size reached approximately 2000 mm³ or when there are animal health issues (40% area of a tumor ulcerated), animals were euthanized, and date of death recorded. Solid tumor volumes were estimated from two-dimensional tumor measurements and calculated according to the following equation:

$$Tumorvolume ( {mm}^{3} ) = \frac{length ( mm ) \times {width}^{2} ( {mm}^{2} )}{2}$$

### Toxicity End Points:

A dosage producing either 15% body weight loss during 3 consecutive days for an individual mouse, 20% body weight loss during 1 day, or 10% or more drug related deaths, was considered an excessively toxic dosage, unless under certain circumstances bodyweight loss or animal death can be considered non-drug related. Examples include animal handling issues such as misgavage, tumor model related issues such as tumor induced cachexia leading to body weight loss that can be observed in control or vehicle treated groups, and excessive tumor ulceration. Mice that had non-drug related death or significant bodyweight loss were not considered toxic and were excluded from statistical analysis. Animal body weight included the tumor weight.

### Efficacy End Points:

The primary efficacy end points include tumor volume changes from baseline summarized by the ratio of medians of tumor volume changes from baseline between the treated and control groups (ΔT/ΔC). Changes in tumor volume for each treated (T) and control (C) group were calculated for each animal on each day by subtracting the tumor volume on the day of first treatment (staging day) from the tumor volume on the specified observation day. The median ΔT was calculated for the treated group and the median ΔC was calculated for the control group. The ratio ΔT/ΔC was calculated and expressed as percentage:

$$Δ T / Δ C = ( \frac{MediandeltaT}{MediandeltaC} ) \times 100$$

ΔT/ΔC≤40% is considered as therapeutically active, ΔT/ΔC=0% is considered as tumor stasis, and ΔT/ΔC<0% is considered as tumor regression (very active). ΔT/ΔC>40% is considered as therapeutically inactive.

Percent tumor regression is defined as % (percentage) of tumor volume decrease in the treated group on a specified observation day compared to its volume when the study was initiated. At a specific time point (t) and for each animal, the regression percentage was calculated using the following formula:

$$\% regression ( at t ) = ( \frac{{volume}_{t 0} - {volume}_{t}}{{volume}_{t 0}} ) \times 100$$

The median percent regression for a group on a given day was then calculated by taking the median of individual % regression values calculated for each animal in the group. The day of calculation was determined by the day when ΔT/ΔC was calculated, except if median percent regression was not representative of the activity of the group. In this case, the day was determined by the first day when the median percent regression was maximal.

### 1-3: Statistical Analysis

A two-way analysis of variance (ANOVA) with factors treatment and day (repeated) was performed on tumor volume changes from baseline. It was followed by contrast analyses with Bonferroni-Holm correction for multiplicity to compare all treated groups to the control group and to compare the combination versus each single agent at the dose involved in the combination at each day from day 0 to 22.

In the figures, the medians and Median Absolute Deviation (MAD) of each group are represented for each day of measurement.

In the tables, the medians and Normalized MAD (nMAD=1.4826*MAD) of each group are reported for each day of measurement.

Tumor volume changes from baseline were calculated for each animal and each day by subtracting the tumor volume on the day of first treatment (day 0) from the tumor volume on the specified observation day.

All statistical analyses were performed using SAS version 9.2 software. A probability of less than 5% (p<0.05) was considered as significant.

### 2: Results

Amcenestrant at 20 mg/kg BID, ribociclib 100 mg/kg QD, and the combination of amcenestrant and ribociclib at the doses and regime for 22 days were well-tolerated, and no significant body weight loss was observed in the study.

Amcenestrant at a dose of 20 mg/kg BID for 22 days had no statistically significant anti-tumor effect on tumor growth with ΔT/ΔC value of 47% (p=0.9411) on day 22. Ribociclib at a dose of 100 mg/kg QD for 22 days induced no statistically significant anti-tumor efficacy with ΔT/ΔC value of 40% (p=0.9411) on day 22. When amcenestrant at 20 mg/kg combined with ribociclib 100 mg/kg with the same dose regime as BID for amcenestrant and QD for ribociclib, the combination treatment demonstrated statistically significant anti-tumor efficacy (tumor stasis) with ΔT/ΔC value of 10% (p<0.0001) on day 22. The statistical analysis indicated that the combination effect was significantly different when compared to either amcenestrant or ribociclib alone on day 22 (p<0.0001 or 0.0012).

Detailed results are shown in Tables 1 to 3 below, as well as in FIGS. 1 and 2.

### BRIEF DESCRIPTION OF THE DRAWINGS

FIG. 1: Antitumor activity of amcenestrant combined with ribociclib against subcutaneous human breast cancer cell line MCF7-Y537S xenograft in nude mice: tumor volume evolution. The curves represent medians +or −MAD (Median Absolute Deviation) at each day for each group.

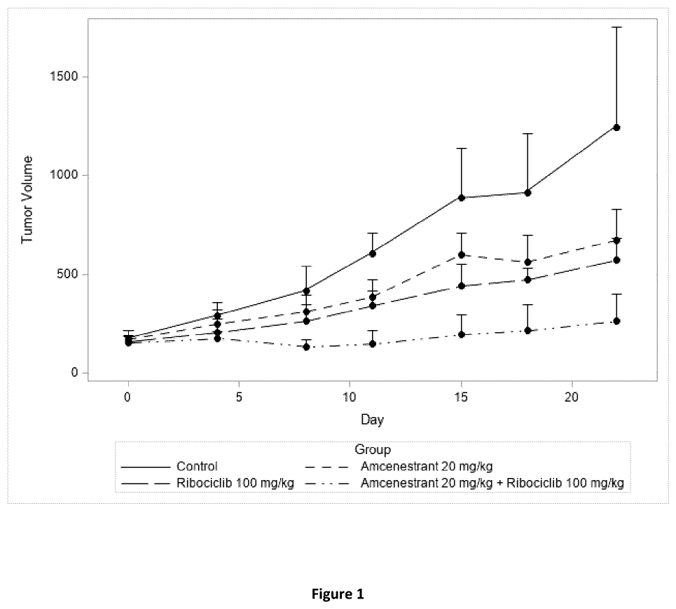

FIG. 2: Antitumor activity of amcenestrant combined with ribociclib against subcutaneous human breast cancer cell line MCF7-Y537S xenograft in nude mice: tumor volume changes from baseline on day 22. Points represent individual tumor volume changes from baseline on day 22, bars correspond to medians.

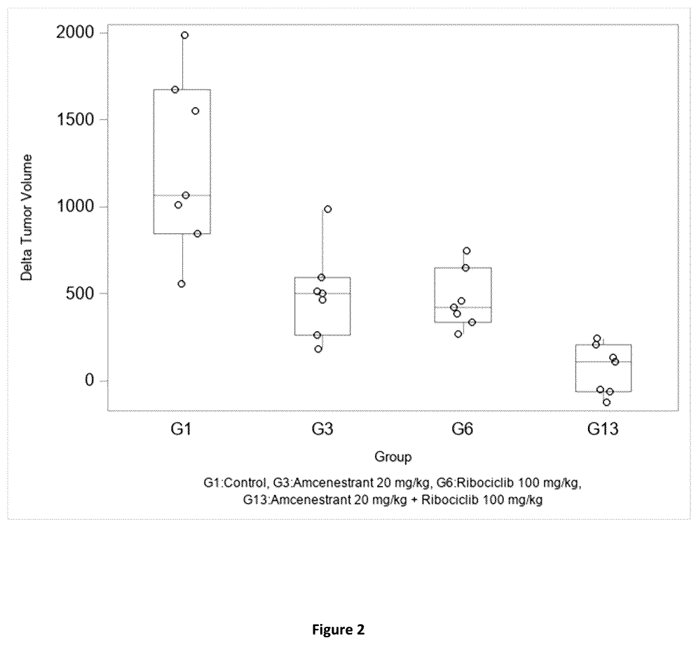

From this experiment, we concluded that amcenestrant at 20 mg/kg twice a day combined with the CDK4/6 inhibitor ribociclib at 100 mg/kg once a day in MCF7-Y537S human breast cancer cell line xenograft model in nude mice induced significant anti-tumor efficacy that was superior to single agents alone, and induced tumor growth inhibition and tumor stasis.

TABLE 1
Efficacy of amcenestrant (SAR439859) combined with ribociclib against
subcutaneous MCF7-Y537S human breast cancer xenograft in nude mice.

| | Route/ | | Schedule | | | | | | | |
|---|---|---|---|---|---|---|---|---|---|---|
| | Dosage (in | Dosage in | in days | *Unscheduled | | Median % of | | | | Biological |
| | mL/kg per | mg/kg per | (total of | death | ΔT/ΔC in | regressions | Regressions | | p-value | Interpre- |
| Agent | injection) | injection | 22 days) | (Day of death) | % at day 22 | on day 22 | Partial | Complete | on day 22 | tation |
| Vehicle | PO, BID (10) | — | 1 | 0/7 | 100 | — | 0/7 | 0/7 | — | — |
| SAR439859 | PO, BID (10) | 20 | 0 to 22 | 0/7 | 47 | 0 | 0/7 | 0/7 | p = 0.9411 | Inactive* |
| ribociclib | PO, QD (10) | 100 | 0 to 22 | 0/7 | 40 | 0 | 0/7 | 0/7 | p = 0.9411 | Inactive* |
| SAR439859 + | PO, BID (10) | 20 + | 0 to 22 | 0/7 | 10 | 0 | 0/7 | 0/7 | p < 0.0001 | Active |
| ribociclib | PO, QD (10) | 100 | | | | | | | | |

PO: per os
*Statistical analysis shows no significant difference (p = 0.9411)

TABLE 2
Efficacy of amcenestrant (SAR439859) combined with ribociclib against
subcutaneous human breast cancer cell line MCF7-Y537S xenograft model in nude mice.
Comparison of each group to the control group at each day.
Tumor volume changes from baseline mm³: Median (nMAD)*, n and p-value^{#}

| Treatment | | | | | | | |
|---|---|---|---|---|---|---|---|
| Group | Global | Day 4 | Day 8 | Day 11 | Day 15 | Day 18 | Day 22 |
| Control | — | 87.0 | 237.0 | 430.0 | 713.0 | 738.0 | 1065.0 |
| | | (25.20) | (105.26) | (75.61) | (272.80) | (382.51) | (717.58) |
| | | n = 7 | n = 7 | n = 7 | n = 7 | n = 7 | n = 7 |
| SAR439859 | — | 43.0 | 121.0 | 214.0 | 360.0 | 392.0 | 501.0 |
| 20 mg/kg | | (51.89) | (45.96) | (69.68) | (220.91) | (100.82) | (131.95) |
| | | n = 7 | n = 7 | n = 7 | n = 7 | n = 7 | n = 7 |
| | 0.2279 | 0.5181 | 0.2234 | 0.1776 | 0.3604 | 0.4047 | 0.9411 |
| ribociclib | — | 44.0 | 103.0 | 183.0 | 292.0 | 348.0 | 423.0 |
| 100 mg/kg | | (38.55) | (75.61) | (88.96) | (133.43) | (126.02) | (126.02) |
| | | n = 7 | n = 7 | n = 7 | n = 7 | n = 7 | n = 7 |
| | 0.2279 | 0.2793 | 0.1929 | 0.1829 | 0.3604 | 0.4133 | 0.9411 |
| SAR439859 | — | −7.0 | −17.0 | −6.0 | 39.0 | 60.0 | 108.0 |
| 20 mg/kg + | | (35.58) | (31.13) | (74.13) | (109.71) | (160.12) | (197.19) |
| ribociclib | | n = 7 | n = 7 | n = 7 | n = 7 | n = 7 | n = 7 |
| 100 mg/kg | <.0001 | 0.0057 | <.0001 | <.0001 | <.0001 | <.0001 | <.0001 |

^{#}p-values obtained with a contrast analysis versus control at each day with Bonferroni-Holm adjustment for multiplicity after a two-way ANOVA on tumor volume changes from baseline.
*MAD = Median Absolute Deviation; nMAD = normalized MAD; nMAD = 1.4826*MAD.
For the combination amcenestrant at 20 mg/kg + ribociclib at 100 mg/kg, the effect on tumor volume changes from baseline is significant compared to the control group from day 4 to day 22.
n = number of animals.

TABLE 3
Efficacy of amcenestrant (SAR439859) combined with ribociclib against subcutaneous
human breast cancer cell line MCF7-Y537S xenograft model in nude mice. Comparison
of amcenestrant 20 mg/kg and ribociclib 100 mg/kg as single agents versus the
combination at each day.
Tumor volume changes from baseline mm³: Median (nMAD)*, n and p-value^{#}

| Treatment | | | | | | | |
|---|---|---|---|---|---|---|---|
| Group | Global | Day 4 | Day 8 | Day 11 | Day 15 | Day 18 | Day 22 |
| SAR439859 | — | −7.0 | −17.0 | −6.0 | 39.0 | 60.0 | 108.0 |
| 20 mg/kg + | | (35.58) | (31.13) | (74.13) | (109.71) | (160.12) | (197.19) |
| ribociclib | | n = 7 | n = 7 | n = 7 | n = 7 | n = 7 | n = 7 |
| 100 mg/kg | | | | | | | |
| ribociclib | — | 44.0 | 103.0 | 183.0 | 292.0 | 348.0 | 423.0 |
| 70 mg/kg | | (38.55) | (75.61) | (88.96) | (133.43) | (126.02) | (126.02) |
| | | n = 7 | n = 7 | n = 7 | n = 7 | n = 7 | n = 7 |
| | <.0001 | 0.9993 | 0.0003 | <.0001 | <.0001 | <.0001 | <.0001 |
| SAR439859 | — | 43.0 | 121.0 | 214.0 | 360.0 | 392.0 | 501.0 |
| 20 mg/kg | | (51.89) | (45.96) | (69.68) | (220.91) | (100.82) | (131.95) |
| | | n = 7 | n = 7 | n = 7 | n = 7 | n = 7 | n = 7 |
| | <.0001 | 0.5762 | <.0001 | <.0001 | <.0001 | <.0001 | <.0001 |

^{#}p-values obtained with a contrast analysis to compare the combinations of amcenestrant and ribociclib versus each single agent at the dose involved in the combination at each day with Bonferroni-Holm adjustment for multiplicity after a two-way ANOVA on tumor volume changes from baseline.
*MAD = Median Absolute Deviation; nMAD = normalized MAD; nMAD = 1.4826*MAD
The effect of the combination of amcenestrant at 20 mg/kg + ribociclib at 100 mg/kg is significantly greater than the effect of ribociclib at 100 mg/kg alone on day 4 to day 22.
The effect of the combination of amcenestrant at 20 mg/kg + ribociclib at 100 mg/kg is significantly greater than the effect of amcenestrant at 20 mg/kg alone on day 4 to day 22.
n = number of animals.

1-17. (canceled)

18. A combination comprising amcenestrant or a pharmaceutically acceptable salt thereof, and ribociclib or a pharmaceutically acceptable salt thereof.

19. The combination according to claim 18, wherein the ribociclib or a pharmaceutically acceptable salt thereof comprises ribociclib succinate.

20. The combination according to claim 18, wherein the combination shows therapeutic synergy.

21. A method of treating cancer comprising administering to a patient in need thereof a therapeutically effective amount of amcenestrant or a pharmaceutically acceptable salt thereof, in combination with a therapeutically effective amount of ribociclib or a pharmaceutically acceptable salt thereof.

22. The method according to claim 21, wherein the cancer is breast cancer.

23. The method according to claim 21, wherein amcenestrant or a pharmaceutically acceptable salt thereof, and ribociclib or a pharmaceutically acceptable salt thereof, are administered separately, either simultaneously or sequentially.

24. The method according to claim 23, wherein the amcenestrant or a pharmaceutically acceptable salt thereof, is administered simultaneously with ribociclib or a pharmaceutically acceptable salt thereof.

25. The method according to claim 23, wherein the amcenestrant or a pharmaceutically acceptable salt thereof, is administered sequentially with ribociclib or a pharmaceutically acceptable salt thereof.

26. A pharmaceutical composition comprising amcenestrant or a pharmaceutically acceptable salt thereof, ribociclib or a pharmaceutically acceptable salt thereof, and at least one pharmaceutically acceptable excipient.

27. The pharmaceutical composition according to claim 26, wherein the ribociclib or a pharmaceutically acceptable salt thereof, comprises ribociclib succinate.

28. A method of treating cancer comprising administering to a patient in need thereof the pharmaceutical composition according to claim 27.

29. The method according to claim 28, wherein the cancer is breast cancer.

30. A method of treating cancer in a patient who is being treated with amcenestrant or a pharmaceutically acceptable salt thereof, comprising administering to the patient an effective amount of ribociclib or a pharmaceutically acceptable salt thereof.

31. A method of treating cancer in a patient on stable treatment with amcenestrant or a pharmaceutically acceptable salt thereof, comprising administering to the patient a therapeutically effective amount of ribociclib or a pharmaceutically acceptable salt thereof.

32. A method of treating cancer comprising administering to a patient in need thereof a therapeutically effective amount of ribociclib or a pharmaceutically acceptable salt thereof, wherein said patient is also being treated with amcenestrant or a pharmaceutically acceptable salt thereof.

33. A kit comprising:
(i) a first pharmaceutical composition comprising amcenestrant or a pharmaceutically acceptable salt thereof, and at least one pharmaceutically acceptable excipient; and
(ii) a second pharmaceutical composition comprising ribociclib or a pharmaceutically acceptable salt thereof, and at least one pharmaceutically acceptable excipient;
wherein the first pharmaceutical composition and the second pharmaceutical composition are in separate compartments.

34. The kit of claim 33, wherein the ribociclib or a pharmaceutically acceptable salt thereof comprises ribociclib succinate.

35. The kit of claim 33, further comprising a package insert indicating that the first and second pharmaceutical compositions are administered simultaneously or sequentially with respect to each other.

36. The kit of claim 33, further comprising a package insert indicating that the first and second pharmaceutical compositions are administered for the treatment of cancer.

37.The kit of claim 36, wherein the cancer is breast cancer.
